(19) United States
(12) Patent Application Publication
(10) Pub. No.: US 2006/0275242 A1
(43) Pub. Date: Dec. 7, 2006

(54) Compositions containing selenium disulfide, a washing base and optionally at least one ether containing two fatty chains, and cosmetic treatment process

(75) Inventors: Sandrine Decoster, Saint-Gratien (FR); Sabina Meralli, Vanves (FR)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/392,561

(22) Filed: Mar. 30, 2006

Related U.S. Application Data
(60) Provisional application No. 60/670,662, filed on Apr. 13, 2005.
(60) Provisional application No. 60/670,661, filed on Apr. 13, 2005.

(30) Foreign Application Priority Data
Mar. 31, 2005 (FR) 05 03154
Mar. 31, 2005 (FR) 05 03153

(51) Int. Cl. A61K 8/46; A61K 33/04

(52) U.S. Cl. 424/70.21; 424/70.31; 424/702

(57) ABSTRACT

Composition containing, in an aqueous medium, a washing base, optionally at least one ether containing two fatty chains, and selenium disulfide, and have a rheological profile defined by a relaxation time of from 1 to 18 ms, measured at 25° C.

### REFERENCE TO PRIOR APPLICATIONS

This application claims priority to U.S. provisional applications 60/670,661 and 60/670,662 both filed Apr. 13, 2005, and to French patent applications 0503154 and 0503153 both filed Mar. 31, 2005, all incorporated herein by reference.

### FIELD OF THE INVENTION

The present invention relates to cosmetic or dermatological compositions comprising selenium disulfide, a washing base and optionally at least one particular ether containing two fatty chains, and to cosmetic processes for treating the hair using the said cosmetic compositions.

### BACKGROUND OF THE INVENTION

To combat the formation of dandruff, which is generally accompanied by microbial and/or fungal proliferation, among the anti-dandruff products that have been proposed are either products that inhibit microbial proliferation or keratolytic products. Among the latter products, the use of selenium disulfide has been most particularly recommended on account of its powerful cytostatic activity (see “Science des traitements capillaires [Hair treatment science], Ch. Zviak, 1988, page 98).

However, although selenium disulfide has excellent anti-dandruff activity, it nevertheless has the drawback of gradually darkening over time, changing from orange to green-brown.

In order to overcome this problem of colour change, document U.S. Pat. No. 4,854,333 has proposed combining selenium disulfide with an oxidizing agent of the peroxide or persalt type. However, the use of oxidizing agents may present problems of toxicity and/or compatibility in the compositions.

Another solution to the problem of changing of colour was proposed in EP 717 981. It consists in combining selenium disulfide with a zinc sulfide of a mineral or organocarboxylic acid to obtain compositions whose colour is stable over time.

However, the presence of zinc salts may be detrimental to the adjustment of the viscosity of the compositions and to the deposition of the conditioning agents that may be present in the compositions.

Within the meaning of the present invention, the term “composition of stable colour” means a composition which, when stored for at least one month in an oven at 45° C., does not show any substantial change in colour compared with its initial colour.

In addition, selenium disulfide has the drawback of leading to shampoo formulations that have mediocre working qualities, and especially difficulty in spreading on the hair and the scalp, which may lead to only moderate efficacy of the antidandruff treatment. It is also relatively unstable in compositions.

In order to overcome the problem of stability in shampoo formulations, document U.S. Pat. No. 4,885,107 has proposed combining selenium disulfide with an alkanolamide and a suspension agent of the long-chain derivative or xanthan gum type. The long-chain derivative may be an ethylene glycol diester, a fatty acid alkanolamide, a long-chain fatty acid ester, a glyceryl ester or a (C₁₆-C₂₂)alkyldimethyl-amine oxide.

### SUMMARY OF THE INVENTION

In order to overcome the drawbacks of the prior art, the inventors have now discovered, surprisingly, that by formulating selenium disulfide in compositions with a particular rheological profile characterized by a relaxation time of from 1 to 18 ms, measured at 25° C., it is possible to obtain better distribution on the hair and the scalp than with the products available on the market, such as those sold under the brand names Dercos® and Head and Shoulders®, and thus better efficacy as regards the anti-dandruff treatment.

In addition, this combination leads to good working qualities such as an abundant and airy lather, rapid development of the lather, and ease and speed of rinsing, and also to good cosmetic properties on the hair, such as softness and smoothness.

Furthermore, the combination of selenium disulfide with a particular ether containing two fatty chains also makes it possible to obtain a composition whose colour is stable over time during its storage.

This colour stability may also be improved by adding at least one fatty alcohol containing at least 18 carbon atoms.

Another advantage of the compositions according to the invention is that their viscosity may readily be adjusted by using standard thickeners.

The cosmetic properties obtained with the compositions according to the invention are also good.

### DETAILED DESCRIPTION OF THE PREFERRED EMBODIMENTS

One subject of the present invention is thus a cosmetic or dermatological composition comprising, in an aqueous medium, a washing base and selenium disulfide, and having a rheological profile defined by a relaxation time of from 1 to 18 ms, measured at 25° C.

A subject of the invention is also a cosmetic or dermatological composition comprising, in an aqueous medium, selenium disulfide, a washing base and at least one ether containing two fatty chains as described below.

Another subject is the use of at least one ether containing two fatty chains and of at least one fatty alcohol containing at least 18 carbon atoms, in a composition containing selenium disulfide, to obtain a rheological profile defined by a relaxation time of from 1 to 18 ms, measured at 25° C.

Other subjects of the invention are cosmetic treatment processes using the said compositions.

Other subjects, characteristics, aspects and advantages of the invention will emerge even more clearly on reading the description and the examples that follow.

According to the invention, the cosmetic or dermatological composition comprises, in an aqueous medium, a washing base and selenium disulfide, and has a Theological profile defined by a relaxation time of from 1 to 18 ms, measured at 25° C.

The relaxation time corresponds to the inverse of the frequency of the crossover point between the elastic modulus (G′) and the viscous modulus (G″).

It may be measured using a Thermo RS600 rotary rheometer equipped with a body of cone-plate geometry. The temperature was maintained at 25° C.

Preferably, the composition according to the invention comprises at least one ether containing two fatty chains as described below.

Another subject of the invention is a cosmetic or dermatological composition comprising, in an aqueous medium:

selenium disulfide,

at least one ether containing two fatty chains as described below, and

a washing base.

The compositions according to the invention, especially in the form of shampoos, thus obtained then have good working qualities, i.e. an abundant and airy lather, rapid development of the lather, and ease and speed of rinsing, but also very good cosmetic properties on the hair, such as softness and smoothness.

The selenium disulfide used in the present invention essentially comprises one selenium atom and two sulfur atoms. It may also be in the form of a polysulfide of structure SeₓS_{y} in which x+y=8.

The selenium disulfide is in the form of a powder with a particle size of less than 200 μm and preferably less than 25 μm.

The selenium disulfide is preferably present in the compositions of the invention in a proportion ranging from 0.001% to 10% by weight, better still from 0.1% to 5% by weight and even more preferentially from 0.2% to 2% by weight relative to the total weight of the composition.

The ethers containing two fatty chains used in the present invention are chosen from ethers containing two fatty chains, which are solid at a temperature equal to about 30° C., corresponding to the following formula:
R—O—R′ (I)
in which:

R and R′, which may be identical or different, denote a linear or branched, saturated or unsaturated hydrocarbon-based group such as alkyl or alkenyl, containing from 12 to 30 carbon atoms and preferably from 14 to 24 carbon atoms, R and R′ being chosen such that the compound of formula (I) is solid at a temperature equal to about 30° C.

Preferably, R and R′ are identical and denote an alkyl group such as stearyl.

The ethers containing two fatty chains that may be used according to the invention are insoluble in cosmetic or dermatological compositions, and may be prepared according to the process described in patent application DE 41 27 230.

The distearyl ether sold under the name Cutina® STE by the company Cognis is especially used in the context of the present invention.

The ether containing two fatty chains is preferably present in a proportion of greater than or equal to 0.5% by weight, better still from 1% to 5% by weight and even more preferentially from 1.3% to 2% by weight relative to the total weight of the composition.

The washing base consists of at least one surfactant chosen from anionic, nonionic and amphoteric or zwitterionic surfactants as described below.

The anionic surfactants that may be used in the composition are especially chosen from salts, in particular alkali metal salts such as sodium salts, ammonium salts, amine salts, amino alcohol salts or alkaline-earth metal salts, for example magnesium salts, of the following types: alkyl sulfates, alkyl ether sulfates, alkylamido ether sulfates, alkylarylpolyether sulfates, monoglyceride sulfates; alkyl sulfonates, alkylamide sulfonates, alkylaryl sulfonates, α-olefin sulfonates, paraffin sulfonates, alkyl sulfosuccinates, alkyl ether sulfosuccinates, alkylamide sulfosuccinates, alkyl sulfoacetates, acyl sarcosinates and acyl glutamates, the alkyl and acyl groups of all these compounds containing from 6 to 24 carbon atoms, and the aryl group preferably denoting a phenyl or benzyl group.

It is also possible to use monoesters of C₆₋₂₄ alkyl and of polyglycoside-dicarboxylic acids, such as alkyl glucoside citrates, polyalkyl glycoside tartrates and polyalkyl glycoside sulfosuccinates, alkyl sulfosuccinamates, acyl isethionates and N-acyltaurates, the alkyl or acyl group of all these compounds containing from 12 to 20 carbon atoms.

Another group of anionic surfactants that may be used in the compositions of the present invention is that of acyl lactylates in which the acyl group contains from 8 to 20 carbon atoms.

In addition, mention may also be made of alkyl-D-galactosiduronic acids and salts thereof, and also polyoxyalkylenated (C₆₋₂₄ alkyl)ether-carboxylic acids, polyoxyalkylenated (C₆₋₂₄ alkyl)(C₆₋₂₄ aryl)ether-carboxylic acids and polyoxyalkylenated (C₆₋₂₄ alkyl)amidoether-carboxylic acids and salts thereof, in particular those comprising from 2 to 50 ethylene oxide units, and mixtures thereof.

Alkyl sulfates and alkyl ether sulfates, and mixtures thereof, are preferably used, in particular in the form of alkali metal, alkaline-earth metal, ammonium, amine or amino alcohol salts.

When they are present, the amount of the anionic surfactant(s) is preferably within the range from 2.5% to 50% by weight and better still from 4% to 20% by weight relative to the total weight of the composition.

The nonionic surfactants that may be used in the compositions of the present invention are compounds that are well known per se (see in particular in this respect “Handbook of Surfactants” by M. R. Porter, published by Blackie & Son (Glasgow and London), 1991, pp. 116-178). They are chosen in particular from polyethoxylated, polypropoxylated or polyglycerolated fatty acids, (C₁-C₂₀) alkylphenols, α-diols or alcohols having a fatty chain containing, for example, 8 to 18 carbon atoms, it being possible for the number of ethylene oxide or propylene oxide groups to range in particular from 2 to 50 and for the number of glycerol groups to range in particular from 2 to 30.

Mention may also be made of condensates of ethylene oxide and of propylene oxide with fatty alcohols; polyethoxylated fatty amides preferably having from 2 to 30 ethylene oxide units, polyglycerolated fatty amides containing on average 1 to 5, and in particular 1.5 to 4, glycerol groups; ethoxylated fatty acid esters of sorbitan having from 2 to 30 mol of ethylene oxide; fatty acid esters of sucrose, fatty acid esters of polyethylene glycol, (C₆₋₂₄ alkyl)polyglycosides, N—(C₆₋₂₄ alkyl)glucamine derivatives, amine oxides such as (C₁₀-C₄)alkylamine oxides or N—(C₁₀₋₁₄ acyl)amino-propylmorpholine oxides.

The amphoteric or zwitterionic surfactants that may be used in the present invention may especially be aliphatic secondary or tertiary amine derivatives in which the aliphatic group is a linear or branched chain containing from 8 to 22 carbon atoms and containing at least one anionic group (for example carboxylate, sulfonate, sulfate, phosphate or phosphonate); mention may also be made of (C₈-C₂₀)alkylbetaines, sulfobetaines, (C₈-C₂₀)alkylamido(C₆-C₈)alkylbetaines or (C₈-C₂₀)alkylamido(C₆-C₈)alkylsulfobetaines.

Among the amine derivatives, mention may be made of the products sold under the name Miranol®, as described in patents U.S. Pat. No. 2,528,378 and U.S. Pat. No. 2,781,354 and classified in the CTFA dictionary, 3rd edition, 1982, under the names Amphocarboxyglycinate and Amphocarboxypropionate, having the respective structures (II) and (III):
Rₐ—CONHCH₂CH₂—N(R_{b})(R_{c})(CH₂COO⁻) (II)

in which:

Rₐ represents an alkyl group derived from an acid Rₐ—COOH present in hydrolysed coconut oil, a heptyl, nonyl or undecyl group,

R_{b} represents a β-hydroxyethyl group, and

R_{c} represents a carboxymethyl group;

and
Rₐ′—CONHCH₂CH₂—N(B)(B′) (III)

in which:

B represents —CH₂CH₂OX′,

B′ represents —(CH₂)_{z}—Y′, with z=1 or 2,

X′ represents a —CH₂CH₂—COOH group or a hydrogen atom,

Y′ represents —COOH or a —CH₂—CHOH—SO₃H group,

Rₐ′ represents an alkyl group of an acid Rₐ′—COOH present in coconut oil or in hydrolysed linseed oil, an alkyl group, in particular a C₁₇ alkyl group and its iso form, an unsaturated C₁₇ group.

These compounds are classified in the CTFA dictionary, 5th edition, 1993, under the names disodium cocoamphodiacetate, disodium lauroamphodiacetate, disodium caprylamphodiacetate, disodium caprylamphodiacetate, disodium cocoamphodipropionate, disodium lauroamphodipropionate, disodium caprylamphodipropionate, disodium caprylamphodipropionate, lauroamphodipropionic acid, cocoamphodipropionic acid.

By way of example, mention may be made of the cocoamphodiacetate sold by the company Rhodia under the trade name Miranol® C2M concentrate.

Among the amphoteric or zwitterionic surfactants mentioned above that are preferably used are (C₈₋₂₀ alkyl)betaines and (C₈₋₂₀ alkyl)amido(C₆₋₈ alkyl)betaines, and mixtures thereof.

When they are present, the amount of the amphoteric or zwitterionic surfactant(s) is preferably within the range from 0.2% to 20% by weight and better still from 0.5% to 10% by weight relative to the total weight of the composition.

Advantageously, the washing base consists of at least one anionic surfactant and at least one amphoteric or zwitterionic surfactant. In a particular embodiment of the present invention the washing base comprises, and more preferably consists of, at least one anionic surfactant, at least one nonionic surfactant and at least one amphoteric or zwitterionic surfactant. Another particular washing base comprises, and more preferably consists of, at least one anionic surfactant and at least one nonionic surfactant.

The amount of the washing base is preferably within the range from 4% to 50% by weight and better still from 4% to 20% by weight relative to the total weight of the composition.

The compositions according to the invention may also comprise at least one fatty alcohol containing at least 18 carbon atoms, preferably from 18 to 30 carbon atoms and even more preferentially from 18 to 24 carbon atoms.

Examples of such fatty alcohols that may especially be mentioned include stearyl alcohol, arachidyl alcohol, behenyl alcohol, lignoceryl alcohol, ceryl alcohol and montanyl alcohol, and mixtures thereof, and more particularly behenyl alcohol.

The fatty alcohol is preferably present in a proportion of greater than or equal to 0.5% by weight, better still from 1% to 5% by weight and even more preferentially from 1.3% to 2% by weight relative to the total weight of the composition.

The compositions according to the present invention may also comprise at least one cationic surfactant.

Examples of cationic surfactants that may especially be mentioned include salts of optionally polyoxyalkylenated primary, secondary or tertiary fatty amines; quaternary ammonium salts such as tetraalkylammonium, alkylamidoalkyltrialkylammonium, trialkyl-benzylammonium, trialkylhydroxyalkylammonium or alkylpyridinium chlorides or bromides; imidazoline derivatives; or amine oxides of cationic nature.

When cationic surfactants are present, their amount is preferably within the range from 0.1% to 10% by weight, better still from 0.2% to 5% by weight and even more preferentially from 0.3% to 3% by weight relative to the total weight of the cosmetic composition.

The compositions according to the present invention may also comprise at least one cationic polymer.

The term “cationic polymer” means any polymer containing cationic groups and/or groups that may be ionized into cationic groups.

The cationic polymers that may be used in accordance with the present invention may be chosen from all those already known per se as improving the cosmetic properties of the hair, i.e. especially those described in patent application EP-A-0 337 354 and in French patent applications FR-A-2 270 846, 2 383 660, 2 598 611, 2 470 596 and 2 519 863.

The cationic polymers that are preferred are chosen from those containing units comprising primary, secondary, tertiary and/or quaternary amine groups that either may form part of the main polymer chain or may be borne by a side substituent directly attached thereto.

The cationic polymers used generally have a weight-average molecular mass of greater than 10⁵, preferably greater than 10⁶ and better still between 10⁶ and 10⁸.

Among the cationic polymers that may be mentioned more particularly are polymers of the polyamine, polyamino amide and polyquaternary ammonium type. These are known products.

The polymers of the polyamine, polyamino amide and polyquaternary ammonium type that may be used in the composition of the present invention are those described in French patents 2 505 348 and 2 542 997. Among these polymers, mention may be made of:

(1) homopolymers or copolymers derived from acrylic or methacrylic esters or amides containing an amine function, comprising at least one of the units of the following formulae: 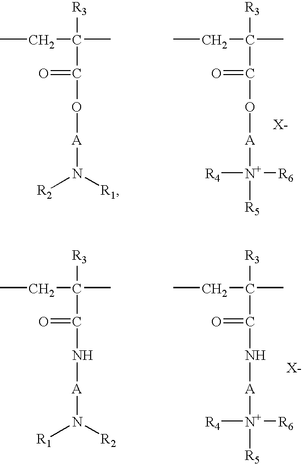
in which:

R₁ and R₂, which may be identical or different, represent a hydrogen atom or an alkyl group containing from 1 to 6 carbon atoms, and preferably a methyl or ethyl group;

R₃, which may be identical or different, denote hydrogen or a CH₃ group;

the symbols A, which may be identical or different, represent a 15 linear or branched alkyl group of 1 to 6 carbon atoms, preferably 2 or 3 carbon atoms, or a hydroxyalkyl group of 1 to 4 carbon atoms;

R₄, R₅ and R₆, which may be identical or different, represent an alkyl group containing from 1 to 18 carbon atoms or a benzyl group and preferably an alkyl group containing from 1 to 6 carbon atoms;

X⁻ denotes an anion derived from a mineral or organic acid, such as a methosulfate anion or a halide such as chloride or bromide.

The copolymers of family (1) can also contain one or more units derived from comonomers which may be chosen from the family of acrylamides, methacrylamides, diacetone acrylamides, acrylamides and methacrylamides substituted on the nitrogen with (C₁-C₄) lower alkyl groups, groups derived from acrylic or methacrylic acids or esters thereof, from vinyllactams such as vinylpyrrolidone or vinylcaprolactam, or from vinyl esters.

Thus, among these copolymers of family (1) that may in particular be mentioned are:

copolymers of acrylamide and of dimethylaminoethyl methacrylate quaternized with dimethyl sulfate or with a dimethyl halide,

the copolymers of acrylamide and of methacryloyloxy-ethyltrimethylammonium chloride described, for example, in patent application EP-A-080 976,

copolymers of acrylamide and of methacryloyloxy-ethyltrimethylammonium methosulfate,

quaternized or non-quaternized vinylpyrrolidone/ dialkyl-aminoalkyl acrylate or methacrylate copolymers. These polymers are described in detail in French patents 2 077 143 and 2 393 573,

dimethylaminoethyl methacrylate/vinylcaprolactam/vinyl-pyrrolidone terpolymers,

vinylpyrrolidone/methacrylamidopropyldimethylamine copolymers, and

quaternized vinylpyrrolidone/dimethylaminopropylmethacryl-amide copolymers.

(2) The cellulose ether derivatives comprising quaternary ammonium groups, which are described in French patent 1 492 597, and in particular the polymers sold under the names “JR” (JR 400, JR 125, JR 30M) or “LR” (LR 400, LR 30M) by the company Union Carbide Corporation. These polymers are also defined in the CTFA dictionary as hydroxyethylcellulose quaternary ammoniums that have reacted with an epoxide substituted with a trimethylammonium group.

(3) Cationic cellulose derivatives such as the copolymers of cellulose or cellulose derivatives grafted with a water-soluble quaternary ammonium monomer, described especially in patent U.S. Pat. No. 4 131 576, such as hydroxyalkylcelluloses, for instance hydroxymethyl-, hydroxyethyl- or hydroxypropylcelluloses grafted especially with a methacryloylethyltrimethylammonium, methacrylamidopropyltri-methylammonium or dimethyldiallylammonium salt.

The commercial products corresponding to this definition are more particularly the products sold under the name Celquat® L 200 and Celquat® H 100 by the company National Starch.

(4) The cationic polysaccharides described more particularly in patents U.S. Pat. No. 3,589,578 and 4,031,307, such as guar gums containing tri-alkylammonium cationic groups. Use is made, for example, of guar gums modified with a salt (e.g. chloride) of 2,3-epoxypropyl-trimethylammonium.

Such products are sold especially under the trade names Jaguar® C13S, Jaguar® C15, Jaguar® C17 and Jaguar® C162 by the company Meyhall.

(5) Polymers consisting of piperazinyl units and of divalent alkylene or hydroxyalkylene radicals containing straight or branched chains, optionally interrupted with oxygen, sulfur or nitrogen atoms or with aromatic or heterocyclic rings, and also the oxidation and/or quaternization products of these polymers. Such polymers are described, in particular, in French patents 2 162 025 and 2 280 361.

(6) Water-soluble polyamino amides prepared in particular by polycondensation of an acidic compound with a polyamine; these polyamino amides can be crosslinked with an epihalohydrin, a diepoxide, a dianhydride, an unsaturated dianhydride, a bis-unsaturated derivative, a bis-halohydrin, a bis-azetidinium, a bis-haloacyldiamine, a bis-alkyl halide or alternatively with an oligomer resulting from the reaction of a difunctional compound which is reactive with a bis-halohydrin, a bis-azetidinium, a bis-haloacyldiamine, a bis-alkyl halide, an epihalohydrin, a diepoxide or a bis-unsaturated derivative; the crosslinking agent is used in proportions ranging from 0.025 to 0.35 mol per amine group of the polyamino amide; these polyamino amides can be alkylated or, if they contain one or more tertiary amine functions, they can be quaternized. Such polymers are described, in particular, in French patents 2 252 840 and 2 368 508.

(7) Polyamino amide derivatives resulting from the condensation of polyalkylene polyamines with polycarboxylic acids followed by alkylation with difunctional agents. Mention may be made, for example, of adipic acid/dialkylaminohydroxyalkyldialkylenetriamine polymers in which the alkyl group contains from 1 to 4 carbon atoms and preferably denotes a methyl, ethyl or propyl group, and the alkylene group contains from 1 to 4 carbon atoms, and preferably denotes an ethylene group. Such polymers are described in particular in French patent 1 583 363.

Among these derivatives, mention may be made more particularly of the adipic acid/dimethylaminohydroxypropyl/diethyl-enetriamine polymers.

(8) Polymers obtained by reaction of a polyalkylene polyamine containing two primary amine groups and at least one secondary amine group with a dicarboxylic acid chosen from diglycolic acid and saturated aliphatic dicarboxylic acids containing from 3 to 8 carbon atoms. The molar ratio between the polyalkylene polyamine and the dicarboxylic acid is between 0.8:1 and 1.4:1; the polyamino amide resulting therefrom is reacted with epichlorohydrin in a molar ratio of epichlorohydrin relative to the secondary amine group of the polyamino amide of between 0.5:1 and 1.8:1. Such polymers are described in particular in U.S. Pat. Nos. 3,227,615 and 2,961,347.

(9) Cyclopolymers of alkyldiallylamine or of dialkyldiallylammonium, such as the homopolymers or copolymers containing, as main constituent of the chain, units corresponding to formula (Va) or (Vb): 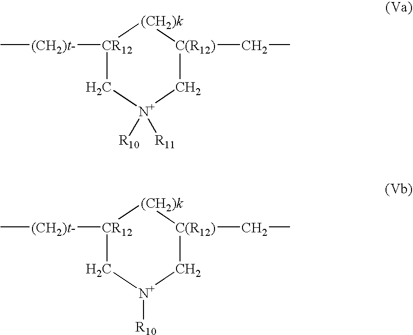
in which k and t are equal to 0 or 1, the sum k+t being equal to 1; R₁₂ denotes a hydrogen atom or a methyl radical; R₁₀ and R₁₁, independently of each other, denote an alkyl group containing from 1 to 6 carbon atoms, a C₁₋₅ hydroxyalkyl group, a lower (C₁-C₄) amidoalkyl group, or R₁₀ and R₁₁, can denote, together with the nitrogen atom to which they are attached, heterocyclic groups such as piperidyl or morpholinyl; Y⁻ is an anion such as bromide, chloride, acetate, borate, citrate, tartrate, bisulfate, bisulfite, sulfate or phosphate. These polymers are described in particular in French patent 2 080 759 and in its Certificate of Addition 2 190 406.

R₁₀ and R₁₁, independently of each other, preferably denote an alkyl group containing from 1 to 4 carbon atoms.

Among the polymers defined above, mention may be made more particularly of the dimethyldiallylammonium chloride homopolymer sold under the name Merquat® 100 by the company Calgon (and its homologues of low weight-average molecular mass) and copolymers of diallyldimethylammonium chloride and of acrylamide, sold under the name Merquat® 550.

(10) The quaternary diammonium polymers containing repeating units corresponding to formula (VI): 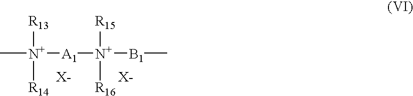
in which:

R₁₃, R₁₄, R₁₅ and R₁₆, which may be identical or different, represent aliphatic, alicyclic or arylaliphatic radicals containing from 1 to 20 carbon atoms or lower hydroxyalkylaliphatic radicals, or alternatively R₁₃, R₁₄, R₁₅ and R₁₆, together or separately, constitute, with the nitrogen atoms to which they are attached, heterocycles optionally containing a second hetero atom other than nitrogen, or alternatively R₁₃, R₁₄, R₁₅ and R₁₆ represent a linear or branched C₁-₆ alkyl radical substituted with a nitrile, ester, acyl or amide group or a group —CO—O—R₁₇-E or —CO—NH—R₁₇-E where R₁₇ is an alkylene group and E is a quaternary ammonium group;

A₁ and B₁, represent polymethylene groups containing from 2 to 20 carbon atoms, which groups may be linear or branched, saturated or unsaturated, and which may contain, linked to or intercalated in the main chain, one or more aromatic rings or one or more oxygen or sulfur atoms or sulfoxide, sulfone, disulfide, amino, alkylamino, hydroxyl, quaternary ammonium, ureido, amide or ester groups, and

X⁻ denotes an anion derived from a mineral or organic acid;

A₁, R₁₃ and R₁₅ can form, with the two nitrogen atoms to which they are attached, a piperazine ring; in addition, if A₁ denotes a linear or branched, saturated or unsaturated alkylene or hydroxyalkylene radical, B₁ can also denote a group:
—(CH₂)ₙ—CO-E′-OC—(CH₂)ₙ—
in which E′ denotes:

a) a glycol residue of formula: —O-Z-O—, where Z denotes a linear or branched hydrocarbon-based radical or a group corresponding to one of the following formulae:
—(CH₂—CH₂—O)ₓ—CH₂—CH₂—
—[CH₂—CH(CH₃)—O]_{y}—CH₂—CH(CH₃)—
where x and y denote an integer from 1 to 4, representing a defined and unique degree of polymerization or any number from 1 to 4 representing an average degree of polymerization;

b) a bis-secondary diamine residue such as a piperazine derivative;

c) a bis-primary diamine residue of formula —NH—Y—NH—, where Y denotes a linear or branched hydrocarbon-based group, or alternatively the divalent group —CH₂—CH₂—S—S—CH₂—CH₂—;

d) a ureylene group of formula —NH—CO—NH—.

Preferably, X⁻ is an anion such as chloride or bromide.

Polymers of this type are described in particular in French patents 2 320 330, 2 270 846, 2 316 271, 2 336 434 and 2 413 907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020.

It is more particularly possible to use polymers that consist of repeating units corresponding to the formula: 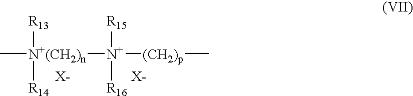
in which R₁₃, R₁₄, R₁₅ and R₁₆, which may be identical or different, denote an alkyl or hydroxyalkyl group containing from 1 to 4 carbon atoms approximately, n and p are integers ranging from 2 to 20 approximately, and X⁻ is an anion derived from a mineral or organic acid.

(11) Polyquaternary ammonium polymers consisting of units of formula (VIII): 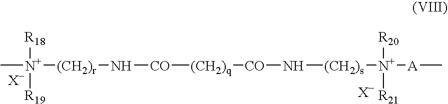
in which:

R₁₈, R₁₉, R₂₀ and R₂₁, which may be identical or different, represent a hydrogen atom or a methyl, ethyl, propyl, β-hydroxyethyl, β-hydroxypropyl or —CH₂CH₂(OCH₂CH₂)ₚOH radical, where p is equal to 0 or to an integer between 1 and 6, with the proviso that R₁₈, R₁₉, R₂₀ and R₂₁ do not simultaneously represent a hydrogen atom,

r and s, which may be identical or different, are integers ranging from 1 to 6,

q is equal to 0 or to an integer ranging from 1 to 34,

X⁻ denotes an anion such as a halide,

A denotes a dihalide radical or preferably represents —CH₂—CH₂—O—CH₂—CH₂—.

Such compounds are described in particular in patent application EP-A-122 324.

(12) Quaternary polymers of vinylpyrrolidone and of vinylimidazole.

(13) Crosslinked or non-crosslinked methacryloyloxy(C₁-₄)alkyltri-(C₁-₄)alkylammonium salt polymers such as the polymers obtained by homopolymerization of dimethylaminoethyl methacrylate quaternized with methyl chloride, or by copolymerization of acrylamide with dimethylaminoethyl methacrylate quaternized with methyl chloride, the homo- or copolymerization being followed by crosslinking with a compound containing olefinic unsaturation, in particular methylenebisacrylamide.

Other cationic polymers which can be used in the context of the invention are cationic proteins or cationic protein hydrolysates, polyalkyleneimines, in particular polyethyleneimines, polymers containing vinylpyridine or vinylpyridinium units, condensates of polyamines and of epichlorohydrin, quaternary polyureylenes and cationic chitin derivatives.

Among all the cationic polymers that may be used in the context of the present invention, it is preferred to use quaternary cellulose ether derivatives such as the products sold under the name JR 400 by the company Union Carbide Corporation, cationic cyclopolymers, in particular the dimethyldiallylammonium chloride homopolymers or copolymers sold under the names Merquat® 100, Merquat® 550 and Merquat® S by the company Calgon, guar gums modified with a 2,3-epoxypropyltrimethylammonium salt, and quaternary polymers of vinylpyrrolidone and of vinylimidazole.

When cationic polymers are present, they are preferably present in an amount ranging from 0.01% to 10% by weight, better still from 0.02% to 5% by weight and even more preferentially from 0.05% to 1% by weight relative to the total weight of the composition.

The compositions according to the invention may also comprise at least one silicone.

The silicones that may be used in accordance with the invention may be soluble or insoluble in the composition. They may be in particular polyorganosiloxanes that are insoluble in the composition and that may be in the form of oils, waxes, resins or gums.

The insoluble silicones are dispersed in the compositions in the form of particles generally with a number-average size of between 2 nanometres and 100 micrometres and preferably between 20 nanometres and 20 micrometres (measured with a granulometer).

The organopolysiloxanes are defined in greater detail in Walter Noll's “Chemistry and Technology of Silicones” (1968) Academic Press. They can be volatile or non-volatile.

When they are volatile, the silicones are more particularly chosen from those having a boiling point of between 60° C. and 260° C., and even more particularly from:

(i) cyclic silicones containing from 3 to 7 and preferably 4 to 5 silicon atoms. These are, for example, octamethylcyclotetrasiloxane sold in particular under the name Volatile Silicone 7207 by Union Carbide or Silbione 70045 V 2 by Rhodia, decamethylcyclo-pentasiloxane sold under the name Volatile Silicone 7158 by Union Carbide, and Silbione 70045 V 5 by Rhodia, and mixtures thereof.

Mention may also be made of cyclocopolymers of the dimethylsiloxanes/methylalkylsiloxane type, such as Volatile Silicone FZ 3109 sold by the company Union Carbide, having the chemical structure: 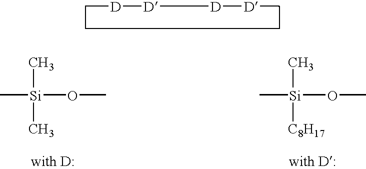
Mention may also be made of mixtures of cyclic silicones with organosilicon compounds, such as the mixture of octamethyl-cyclotetrasiloxane and tetratrimethylsilylpentaerythritol (50/50) and the mixture of octamethylcyclotetrasiloxane and oxy-1,1′-bis(2,2,2′,2′,3,3′-hexatrimethylsilyloxy)neopentane;

(ii) linear volatile silicones containing 2 to 9 silicon atoms and having a viscosity of less than or equal to 5×10⁻⁶ m²/s at 25° C. An example is decamethyltetrasiloxane sold in particular under the name SH 200 by the company Toray Silicone. Silicones belonging to this category are also described in the article published in Cosmetics and Toiletries, Vol. 91, January 76, pp. 27-32, Todd & Byers “Volatile Silicone Fluids for Cosmetics”.

Among the non-volatile silicones that may especially be mentioned are polyalkylsiloxanes, polyalkylsiloxanes, polyalkyl-arylsiloxanes, silicone gums and resins, polyorganosiloxanes modified with organofunctional groups, linear block polysiloxane(A)-polyoxyalkylene (B) copolymers of (A-B)ₙ type with n>3; grafted silicone polymers, containing a non-silicone organic skeleton, consisting of a main organic chain formed from organic monomers not comprising silicone, onto which is grafted, within the said chain and also optionally on at least one of its ends, at least one polysiloxane macromonomer; grafted silicone polymers, with a polysiloxane skeleton grafted with non-silicone organic monomers, comprising a main polysiloxane chain onto which is grafted, within the said chain. and also optionally on at least one of its ends, at least one organic macromonomer not comprising silicone; and also mixtures thereof.

Examples of polyalkylsiloxanes that may especially be mentioned include polydimethylsiloxanes containing trimethylsilyl end groups having a viscosity of from 5×10⁻⁶ to 2.5 m²/s at 25° C. and preferably 1×10⁻⁵ to 1 m²/s. The viscosity of the silicones is measured, for example, at 25° C. according to ASTM standard 445 Appendix C.

Among these polyalkylsiloxanes, mention may be made, in a nonlimiting manner, of the following commercial products:

the Silbione oils of the 47 and 70 047 series or the Mirasil oils sold by Rhône-Poulenc, such as, for example, the oil 70 047 V 500 000;

the oils of the Mirasil series sold by the company Rhône-Poulenc;

the oils of the 200 series from the company Dow Corning, such as, more particularly, DC200 with a viscosity of 60 000 cSt;

the Viscasil oils from General Electric and certain oils of the SF series (SF 96, SF 18) from General Electric.

Mention may also be made of polydimethylsiloxanes containing dimethylsilanol end groups (Dimethiconol according to the CTFA name) such as the oils of the 48. series from the company Rhône-Poulenc.

In this category of polyalkylsiloxanes, mention may also be made of the products sold under the names Abil Wax 9800 and 9801 by the company Goldschmidt, which are poly(C₁-C₂₀)alkylsiloxanes.

The polyalkylarylsiloxanes are chosen particularly from linear and/or branched polydimethylmethylphenylsiloxanes and polydimethyldiphenylsiloxanes with a viscosity of from 1×10⁻⁵ to 5×10⁻² m²/s at 25° C.

Among these polyalkylarylsiloxanes, mention may be made, by way of example, of the products sold under the following names:

the Silbione oils of the 70 641 series from Rhône-Poulenc;

the oils of the Rhodorsil 70 633 and 763 series from Rhône-Poulenc;

the oil Dow Corning 556 Cosmetic Grade Fluid from Dow Corning;

the silicones of the PK series from Bayer, such as the product PK20;

the silicones of the PN and PH series from Bayer, such as the products PN1000 and PH1000;

certain oils of the SF series from General Electric, such as SF 1023, SF 1154, SF 1250 and SF 1265.

The silicone gums that can be used in accordance with the invention are, in particular, polydiorganosiloxanes having high number-average molecular masses of between 200 000 and 1 000 000, used alone or as a mixture in a solvent. This solvent can be chosen from volatile silicones, polydimethylsiloxane (PDMS) oils, polyphenylmethylsiloxane (PPMS) oils, isoparaffins, polyisobutylenes, methylene chloride, pentane, dodecane and tridecanes, or mixtures thereof.

Mention may be made more particularly of the following products:

polydimethylsiloxane,

polydimethylsiloxane/methylvinylsiloxane gums,

polydimethylsiloxane/diphenylsiloxane,

polydimethylsiloxane/phenylmethylsiloxane,

polydimethylsiloxane/diphenylsiloxane/methylvinylsiloxane.

Silicones that can be used in the composition according to the invention are mixtures such as:

mixtures formed from a polydimethylsiloxane hydroxylated at the chain end (referred to as dimethiconol according to the nomenclature in the CTFA dictionary) and from a cyclic polydimethylsiloxane (referred to as cyclomethicone according to the nomenclature in the CTFA dictionary), such as the product Q2 1401 sold by the company Dow Corning;

mixtures formed from a polydimethylsiloxane gum with a cyclic silicone, such as the product SF 1214 Silicone Fluid from the company General Electric; this product is an SF 30 gum corresponding to a dimethicone, having a number-average molecular weight of 500 000, dissolved in the oil SF 1202 Silicone Fluid corresponding to decamethylcyclopentasiloxane;

mixtures of two PDMSs of different viscosities, and more particularly of a PDMS gum and a PDMS oil, such as the product SF 1236 from the company General Electric. The product SF 1236 is a mixture of an SE 30 gum defined above, having a viscosity of 20 m²/s, and an SF 96 oil, with a viscosity of 5×10⁻⁶ m²/s. This product preferably contains 15% SE 20 gum and 85% SF 96 oil.

The organopolysiloxane resins that can be used in accordance with the invention are crosslinked siloxane systems containing the following units:

R₂SiO_{2/2}, R₃SiO_{1/2}, RSiO_{3/2} and SiO_{4/2} in which R represents a hydrocarbon-based group containing 1 to 16 carbon atoms or a phenyl group.

Among these products, those particularly preferred are the ones in which R denotes a C₁-C₄ lower alkyl radical, more particularly methyl, or a phenyl radical.

Among these resins, mention may be made of the product sold under the name Dow Corning 593 or those sold under the names Silicone Fluid SS 4230 and SS 4267 by the company General Electric, which are silicones of dimethyl/trimethyl siloxane structure.

Mention may also be made of the trimethyl siloxysilicate type resins sold in particular under the names X22-4914, X21-5034 and X21-5037 by the company Shin-Etsu.

The organomodified silicones that can be used in accordance with the invention are silicones as defined above and containing in their structure one or more organofunctional groups attached via a hydrocarbon-based radical.

Among the organomodified silicones, mention may be made of polyorganosiloxanes comprising:

polyethyleneoxy and/or polypropyleneoxy groups optionally comprising C₆-C₂₄ alkyl groups, such as the products known as dimethicone copolyol sold by the company Dow Corning under the name DC 1248 or the oils Silwet® L 722, L 7500, L 77 and L 711 by the company Union Carbide, and the (C₁₂)alkylmethicone copolyol sold by the company Dow Corning under the name Q2 5200;

substituted or unsubstituted amine groups, such as the products sold under the name GP 4 Silicone Fluid and GP 7100 by the company Genesee, or the products sold under the names Q2 8220 and Dow Corning 929 or 939 by the company Dow Corning. The substituted amine groups are, in particular, C₁-C₄ aminoalkyl groups;

quaternary ammonium groups, for instance the products sold under the names Abil Quat 3272 and Abil Quat 3474 by the company Goldschmidt;

thiol groups such as the products sold under the names GP 72 A and GP 71 from Genesee;

alkoxylated groups such as the product sold under the name Silicone Copolymer F-755 by SWS Silicones and Abil Wax® 2428, 2434 and 2440 by the company Goldschmidt;

hydroxylated groups such as the polyorganosiloxanes containing a hydroxyalkyl function, described in French patent application FR-A-85/16334;

acyloxyalkyl groups such as, for example, the polyorganosiloxanes described in patent U.S. Pat. No. 4,957,732;

anionic groups of carboxylic type, such as, for example, in the products described in patent EP 186 507 from the company Chisso Corporation, or of alkylcarboxylic type, such as those present in the product X-22-3701E from the company Shin-Etsu; 2-hydroxyalkyl sulfonate; 2-hydroxyalkyl thiosulfate such as the products sold by the company Goldschmidt under the names Abil® S201 and Abil® S255;

hydroxyacylamino groups, such as the polyorganosiloxanes described in patent application EP 342 834. Mention may be made, for example, of the product Q2-8413 from the company Dow Corning.

The silicones that are particularly preferred in the invention are polydimethylsiloxanes such as polydimethylsiloxanes containing trimethylsilyl end groups, or polydimethylsiloxanes containing hydroxydimethylsilyl end groups, and amino silicones.

When the said silicones are present, they are preferably contained in an amount ranging from 0.05% to 20% by weight, more particularly from 0.1% to 10% by weight and better still from 0.5% to 5% by weight relative to the total weight of the composition.

The aqueous medium consists of water or of a mixture of water and of at least one cosmetically or dermatologically acceptable solvent chosen from C₁-C₄ lower alcohols, such as ethanol, isopropanol, tert-butanol or n-butanol; polyols such as glycerol, propylene glycol and polyethylene glycols; and mixtures thereof.

The pH of the compositions according to the invention is generally less than 8.5 and preferably within the range from 4 to 7.

The composition according to the invention may also comprise one or more standard additives that are well known in the art, such as antidandruff agents other than selenium disulfide, hair-loss counteractants, oxidizing agents, ceramides and pseudoceramides, vitamins and provitamins including panthenol, plant, animal, mineral and synthetic oils, waxes, ceramides and pseudoceramides, sunscreens, coloured or colourless, mineral and organic pigments, dyes, nacreous agents and opacifiers, sequestrants, plasticizers, solubilizers, acidifying agents, basifying agents, mineral and organic thickeners, antioxidants, hydroxy acids, fragrances and preserving agents.

A person skilled in the art will take care to select the optional additives and the amount thereof such that they do not harm the properties of the compositions of the present invention.

These additives are generally present in the composition according to the invention in an amount ranging from 0 to 20% by weight relative to the total weight of the composition.

Another subject of the invention is the use of at least one ether containing two fatty chains and of at least one fatty alcohol containing at least 18 carbon atoms as defined above, in a composition containing selenium disulfide, to obtain a rheological profile defined by a relaxation time of from 1 to 18 ms, measured at 25° C.

This use makes it possible also to obtain good working qualities such as an abundant and airy lather, rapid development of the lather, and ease and speed of rinsing, during the application of the composition to the hair and the scalp, and also good cosmetic properties such as softness and smoothness, after application of the composition.

Another subject of the invention is a cosmetic process for treating the hair, which consists in applying an effective amount of a cosmetic composition as described above to the said materials, and rinsing after an optional leave-on time.

The examples that follow are given as illustrations of the present invention.

In the examples that follow, all the amounts are indicated as weight percentages of active material relative to the total weight of the composition, unless otherwise mentioned.

### EXAMPLES

### Example 1

The composition below is prepared from the ingredients indicated in the table below.

| | |
|---|---|
| Sodium lauryl ether sulfate | 14.7% |
| Cocoylbetaine | 2.4% |
| Selenium disulfide | 1% |
| Distearyl ether | 1.5% |
| Mixture of fatty alcohols sold under the brand name | 1.5% |
| Nafol 1822C by the company Sasol | |
| Carboxyvinyl polymer (Carbopol ® 980) | 0.4% |
| Polydimethylsiloxane | 2% |
| (Dow Corning 200 Fluid 60 000 cSt) | |
| Fragrance | 0.5% |
| Citric acid qs | pH 4.8-5.2 |
| Water qs | 100% |

An orange-coloured nacreous gel is obtained.

After two months of storage at 45° C., no change in colour was observed and the antidandruff activity is conserved.

This composition is a shampoo that is applied to wet hair by massaging the scalp. The composition is left on the hair for two minutes and is then rinsed out.

After drying, the hair is soft and detangled. Good antidandruff efficacy is also observed.

The rheological measurements were performed using a Thermo RS600 rotary viscometer equipped with a cone-plate geometry, 60 mm/1° (titanium). The temperature was maintained at 25° C.

The results obtained are collated in the table below.

| | Relaxation time (ms) |
|---|---|
| Composition A | 11 |
| Product Dercos ® containing selenium | 20 |
| disulfide⁽¹⁾ | |
| Product H&S ® containing selenium | 32 |
| disulfide⁽²⁾ | |

⁽¹⁾comprising cetyl alcohol and hydroxystearyl cetyl alcohol.
⁽²⁾comprising glycol distearate.

The products Dercose® and H&S® have a relaxation time outside the range of the invention.

In addition, composition A has better working qualities than the products Dercos® and H&S®.

### Example 2

The composition below is prepared using the ingredients indicated in the table below.

| | |
|---|---|
| Sodium lauryl sulfate | 14.7% |
| Disodium cocoamphodiacetate | 3.5% |
| Selenium disulfide | 0.8% |
| Distearyl ether | 2% |
| Mixture of fatty alcohols sold under the trade name | 2% |
| Nafol 1822C by the company Sasol | |
| Carboxyvinyl polymer (Carbopol ® 980) | 0.4% |
| Polydimethylsiloxane | 2% |
| (Dow Corning 200 Fluid 60 000 cSt) | |
| Fragrance | qs |
| Citric acid qs | pH 4.8-5.2 |
| Water qs | 100% |

### Example 3

The composition below is prepared from the ingredients indicated in the table below.

| | |
|---|---|
| Sodium lauryl ether sulfate containing 2.2 mol of | 10% |
| ethylene oxide (or EO) | |
| Lauryl ether carboxylic acid containing 4.5 mol of | 10% |
| EO | |
| Selenium disulfide | 1.5% |
| Distearyl ether | 1.5% |
| Mixture of fatty alcohols sold under the trade name | 0.9% |
| Nafol 1822C by the company Sasol | |
| Polyvinylpyrrolidone | 0.02% |
| Polydimethylsiloxane | 0.5% |
| (Dow Corning 200 Fluid 60 000 cSt) | |
| Fragrance | qs |
| Citric acid qs | pH 4.8-5.2 |
| Water qs | 100% |

### Example 4

The composition below is prepared from the ingredients indicated in the table below.

| | |
|---|---|
| Sodium lauryl sulfate | 8% |
| Sodium lauryl ether sulfate containing 2.2 mol of | 10% |
| ethylene oxide (or OE) | |
| Cocoamidopropylbetaine | 2.5% |
| Glyceryl laurate | 0.5% |
| Selenium disulfide | 0.5% |
| Distearyl ether | 1.5% |
| Mixture of fatty alcohols sold under the trade name | 1.5% |
| Nafol 1822C by the company Sasol | |
| Cationic polymer Jaguar C13S | 0.02% |
| Carboxyvinyl polymer (Carbopol ® 980) | 0.4% |
| Polydimethylsiloxane | 2% |
| (Dow Corning 200 Fluid 60 000 cSt) | |
| Fragrance | qs |
| Citric acid qs | pH 4.8-5.2 |
| Water qs | 100% |

The above written description of the invention provides a manner and process of making and using it such that any person skilled in this art is enabled to make and use the same, this enablement being provided in particular for the subject matter of the appended claims, which make up a part of the original description and including a cosmetic or dermatological composition comprising, in an aqueous medium, a washing base and selenium disulfide, and having a rheological profile defined by a relaxation time of from 1 to 18 ms, measured at 25° C.

As used above, the phrases “selected from the group consisting of,” “chosen from,” and the like include-mixtures of the specified materials. Terms such as “contain(s)” and the like as used herein are open terms meaning “including at least” unless otherwise specifically noted.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The invention methods and compositions are preferably used by subjects desirous of the benefits noted herein, subjects “in need of” these benefits.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A composition comprising, in an aqueous medium, a washing base and selenium disulfide, said composition having a heological profile defined by a relaxation time of from 1 to 18 ms, measured at 25° C.

2. The composition according to claim 1, wherein it further comprises at least one ether comprising two fatty chains.

3. The composition according to claim 2, comprising at least one ether comprising two fatty chains chosen from ethers which are solid at a temperature equal to about 30° C., corresponding to the following formula:
R—O—R′ (I)
in which:
R and R′, which may be identical or different, denote a linear or branched, saturated or unsaturated hydrocarbon-based group containing from 12 to 30 carbon atoms, R and R′ being chosen such that the compound of formula (I) is solid at a temperature equal to about 30° C.

4. The composition according to claim 2,
comprising at least one ether comprising two fatty chains which is solid at a temperature of less than or equal to about 30° C., corresponding to the following formula:
R—O—R′ (I)
in which:
R and R′, which may be identical or different, denote a linear or branched alkyl or alkenyl group containing from 12 to 30 carbon atoms, R and R′ being chosen such that the compound of formula (I) is solid at a temperature equal to about 30° C.

5. The composition according to claim 3, comprising distearyl ether.

6. The composition according to claim 3, wherein the ether containing two fatty chains is present in an amount of greater than or equal to 0.5% by weight relative to the total weight of the composition.

7. The composition according to claim 6, wherein the ether containing two fatty chains is present in an amount ranging from 1% to 5% by weight relative to the total weight of the composition.

8. The composition according to claim 1, wherein the washing base comprises at least one surfactant chosen from anionic, non-ionic, amphoteric and zwitterionic surfactants.

9. The composition according to claim 8, wherein the washing base comprises at least one anionic surfactant and at least one amphoteric or zwitterionic surfactant, or at least one anionic surfactant and at least one nonionic surfactant, or at least one anionic surfactant, at least one nonionic surfactant, and at least one amphoteric or zwitterionic surfactant.

10. The composition according to claim 8, comprising at least one anionic surfactant chosen from alkyl sulfates and alkyl ether sulfates, and mixtures thereof.

11. The composition according to claim 8, comprising at least one surfactant chosen from (C₈-C₂₀ alkyl)betaines and (C₈-C₂₀ alkyl)amido(C₆-C₈ alkyl)betaines, and mixtures thereof.

12. The composition according to claim 1, wherein the washing base is present in an amount ranging from 4% to 50% by weight relative to the total weight of the composition.

13. The composition according to claim 1, further comprising at least one fatty alcohol containing at least 18 carbon atoms.

14. The composition according to claim 13, wherein the fatty alcohol contains from 18 to 30 carbon atoms.

15. The composition according to claim 13, wherein the fatty alcohol is chosen from stearyl alcohol, arachidyl alcohol, behenyl alcohol, lignoceryl alcohol, ceryl alcohol, montanyl alcohol, and mixtures thereof.

16. The composition according to claim 13, wherein the fatty alcohol is present in an amount of greater than or equal to 0.5% by weight relative to the total weight of the composition.

17. The composition according to claim 16, wherein the fatty alcohol is present in an amount ranging from 1% to 5% by weight relative to the total weight of the composition.

18. The composition according to claim 1, further comprising at least one cationic polymer.

19. The composition according to claim 1, further comprising at least one silicone.

20. The composition according to claim 1, wherein the aqueous medium comprises water and optionally at least one cosmetically or dermatologically acceptable solvent.

21. The composition according to claim 20, wherein the aqueous medium comprises at least one C₁-C₄ lower alcohol and/or C₁-C₄ lower polyol.

22. The composition according to claim 1, further comprising at least one additive chosen from antidandruff agents other than selenium disulfide, hair-loss counteractants, oxidizing agents, ceramides and pseudoceramides, vitamins and provitamins including panthenol, plant, animal, mineral and synthetic oils, waxes, ceramides and pseudoceramides, sunscreens, coloured or colourless, mineral and organic pigments, dyes, nacreous agents and opacifiers, sequestrants, plasticizers, solubilizers, acidifying agents, basifying agents, mineral and organic thickeners, antioxidants, hydroxy acids, fragrances and preserving agents.

23. A method, comprising applying the composition of claim 1 to human hair.

24. The method of claim 23, wherein said hair is wet hair.
